(19) United States
(12) Patent Application Publication
(10) Pub. No.: US 2026/0061248 A1
(43) Pub. Date: Mar. 5, 2026

(54) Intermittent Hypoxic-Hyperoxic Training Device for Both Dynamic and Static Use, Training Method, and Terminal

(71) Applicant: Shanghai Rejuvelab Medical and Health Technology Co., Ltd., Shanghai (CN)

(72) Inventors: Nannan LIAN, Shanghai (CN); Hengyang XI, Shanghai (CN); Jinxin YU, Shanghai (CN); Dawei ZHAO, Shanghai (CN); Haiyun WANG, Shanghai (CN); Cheng PENG, 200433 (CN)

(73) Assignee: Shanghai Rejuvelab Medical and Health Technology Co., Ltd., Shanghai (CN)

(21) Appl. No.: 19/075,761

(22) Filed: Mar. 10, 2025

(30) Foreign Application Priority Data
Aug. 27, 2024 (CN) 2024111840285

(51) Int. Cl. A63B 22/00; A63B 24/00; G16H 20/30

(52) U.S. Cl. CPC A63B 22/00; A63B 24/0062; A63B 24/0087; G16H 20/30; A63B 2214/00

(57) ABSTRACT

An intermittent hypoxic-hyperoxic training device for both dynamic and static use is provided. The training device has multiple training modes. The training device includes a gas generating device, a control device, an interactive display device, a heart rate wearing device, and a blood oxygen saturation detection device. The control device preliminarily sets an oxygen content parameter of a mixed gas upon receiving a corresponding training instruction sent by the interactive display device in response to the training mode selected by the user instruction; a heart rate parameter of a trained object is acquired from the heart rate wearing device, to adjust the oxygen content parameter under an exercise state according to the heart rate parameter; and/or a blood oxygen parameter of the trained object is acquired from the blood oxygen saturation detection device, to adjust the oxygen content parameter under a non-exercise state according to the blood oxygen parameter.

### TECHNICAL FIELD

The present application relates to the technical field of sports fitness apparatus, in particular to an intermittent hypoxic-hyperoxic training device for both dynamic and static use, a training method, and a terminal.

### BACKGROUND

Intermittent hypoxic-hyperoxic training requires simulating a hypoxic and low-pressure environment at high-altitude areas to achieve training effects of promoting cellular perception and adapting to an oxygen change mechanism, so as to effectively improve the body function, and improve the tolerance of the body to the hypoxic environment. At present, as proved by medical science and physiology, the intermittent hypoxic-hyperoxic training can obviously improve hypertension, hyperlipidemia, atherosclerosis, myocardial function, haematopoietic ability and human brain perception of some people, and can be used to improve the body function, and enhance the immune system, non-specific compensation ability, and aerobic output of the body. However, gas control of the current breathing training devices is not fine and flexible enough and cannot be applied to various states of the user, the structure is complicated, the reliability is low, the airflow cannot be output stably, and the gas composition cannot be stably adjusted.

### SUMMARY OF THE INVENTION

The present application provides an intermittent hypoxic-hyperoxic training device for both dynamic and static use, a training method, and a terminal.

A first aspect of the present application provides an intermittent hypoxic-hyperoxic training device for both dynamic and static use. The training device is equipped with multiple training modes, and the training device includes: a training device body, where a gas generating device for generating a mixed gas and a control device are provided inside the training device; an interactive display device arranged on a surface of the training device body to send a corresponding training instruction to the control device in response to a training mode selected by a user instruction to preliminarily set an oxygen content parameter of the mixed gas; a heart rate wearing device electrically connected to the control device, wherein the heart rate wearing device is worn on a trained object for collecting a heart rate parameter of the trained object; a blood oxygen saturation detection device electrically connected to the control device, wherein the blood oxygen saturation detection device is worn on the trained object for collecting a blood oxygen parameter of the trained object; and the control device acquires the heart rate parameter of the trained object from the heart rate wearing device for adjusting the oxygen content parameter under an exercise state according to the heart rate parameter; and/or, the control device acquires the blood oxygen parameter of the trained object from the blood oxygen saturation detection device for adjusting the oxygen content parameter under a non-exercise state according to the blood oxygen parameter.

In some embodiments of a first aspect of the present application, the gas generating device includes: a fluid device, a purifying device, a gas separating device, and a gas mixing device connected in sequence; wherein the fluid device is connected to a gas-liquid separator through a heat exchanger, the purifying device is connected to the gas-liquid separator through the heat exchanger, and the gas-liquid separator is connected to a drainage structure; the gas separating device is provided with a first outlet and a second outlet; the first outlet is connected to the gas mixing device via a first valve, and the second outlet is connected to the gas mixing device via a second valve.

In some embodiments of the first aspect of the present application, the drainage structure includes: a first drainage pipe and a second drainage pipe; wherein the first drainage pipe is provided with a first drainage valve, and the second drainage pipe is provided with a second drainage valve.

In some embodiments of the first aspect of the present application, the training device is further equipped with an external atomizing device; the external atomizing device is connected to the first drainage pipe, the second drainage pipe, and the gas mixing device, respectively, to adjust a humidity parameter of the mixed gas; the external atomizer is internally stored with liquid; and the liquid includes: cordyceps, saline, and aroma.

In some embodiments of the first aspect of the present application, the control device is communicatively connected to the first valve, the second valve, the first drainage valve and the second drainage valve, respectively; and the control device adjusts the oxygen content parameter and the humidity parameter by controlling the first valve, the second valve, the first drainage valve and the second drainage valve.

In some embodiments of the first aspect of the present application, the control device is deployed with a well-trained gas parameter optimization model; and the gas parameter optimization model optimizes use parameters for the trained object based on characteristics and preferences of the trained object.

In some embodiments of the first aspect of the present application, during a training process, the gas parameter optimization model may acquire basic data, exercise habit data, physiological index data, environmental parameter data and historical use parameter data of the trained object, and form a data set of the trained object after pre-processing all the acquired data; and divide the data set of the trained object into a training set, a validation set, and a test set according to a preset ratio; select a machine learning model and initializing model parameters; input the training set into the machine learning model for training, calculate a prediction by forward propagation, and update the model parameters by back propagation; use the validation set to adjust parameters of a model, and evaluate a generalization ability of the model using the test set.

In some embodiments of the first aspect of the present application, the gas parameter optimization model optimizes the use parameters for the trained object based on the characteristics and preferences of the trained object. Specifically, the gas parameter optimization model may identify identity information of the current trained object to acquire characteristics and preferences of the current trained object, input the acquired characteristics and preferences of the current trained object into the well-trained gas parameter optimization model for calculation, and output use parameters applicable to the current trained object; the use parameters include: an oxygen content parameter, training period, total training time, number of periods, rest time, training intensity, training mode, and user interface parameters.

A second aspect of the present application provides an intermittent hypoxic-hyperoxic training method applied to an intermittent hypoxic-hyperoxic training device for both dynamic and static use, wherein the training device is equipped with multiple training modes, the training device includes an interactive display device, a gas generating device, a heart rate wearing device, and a blood oxygen saturation detection device. The method includes: preliminarily setting an oxygen content parameter of a mixed gas upon receiving a corresponding training instruction sent by the interactive display device in response to the training mode selected by the user instruction; and acquiring a heart rate parameter of the trained object from the heart rate wearing device, so as to adjust the oxygen content parameter under an exercise state according to the heart rate parameter; and/or acquiring a blood oxygen parameter of the trained object from the blood oxygen saturation detection device, so as to adjust the oxygen content parameter under a non-exercise state according to the blood oxygen parameter.

A third aspect of the present application provides an electronic terminal including a memory, a processor and a computer program stored on the memory; the processor executes the computer program to implement the above intermittent hypoxic-hyperoxic training method.

As described above, the intermittent hypoxic-hyperoxic training device for both dynamic and static use, the training method, and the terminal of the present application have the following beneficial effects:
(1) In the present application, gas parameters can be adjusted in real time for different states of the user.
(2) In the present application, the gas composition is adjusted flexibly and stably and a stable airflow is output.

### BRIEF DESCRIPTION OF THE DRAWINGS

FIG. 1 shows a structural schematic diagram of an intermittent hypoxic-hyperoxic training device for both dynamic and static use in an embodiment of the present application.

FIG. 2 shows a structural schematic diagram of a gas generating device in an embodiment of the present application.

FIG. 3 shows a schematic diagram of a drainage structure in an embodiment of the present application.

FIG. 4 shows a structural schematic diagram of an external atomizer in an embodiment of the present application.

FIG. 5 shows a schematic flow chart of an intermittent hypoxic-hyperoxic training method in an embodiment of the present application.

FIG. 6 shows a structural schematic diagram of an electronic terminal in an embodiment of the present application.

### DETAILED DESCRIPTION

Embodiments of the present application are described below by means of specific examples, and those skilled in the art can easily understand other advantages and effects of the present application from the contents disclosed in the present specification. The present application may also be implemented or applied through other different specific embodiments, and the details in the present specification may also be modified or changed in various ways based on different views and applications without departing from the spirit of the present application. It should be noted that the following embodiments and features in the embodiments may be combined with each other without conflict.

In the embodiments of the present application, words such as “first” and “second” are used to distinguish the same or similar items with basically the same function and role. For example, the first outlet and the second outlet are merely used for distinguishing different outlets, and do not limit their order of precedence. Those skilled in the art may understand that the words “first”, “second” and the like do not limit the number or the order of execution, and the words “first”, “second” and the like also do not limit that they are necessarily different.

It should be noted that in the embodiments of the present application, the words “exemplary” or “for example” denote examples, illustrations or descriptions. Any embodiment or design solution described as “exemplary” or “for example” in the present application should not be construed as being preferred or advantageous over other embodiments or design solutions. Rather, the use of the words “exemplary” or “for example”is intended to present relevant concepts in a specific manner.

In the embodiments of the present application, “at least one” means one or more, and “a plurality of” or “multiple” means two or more. The word “and/or” which describes an association relationship of associated objects indicates that three relationships may exist, for example, A and/or B, which may indicate: the existence of A alone, the existence of both A and B, and the existence of B alone, wherein A and B may be singular or plural. The character “/” generally indicates that the associated objects are in an “or” relationship. The expression “at least one of the following” or its equivalent refers to any combination of these items, including any combination of singular or plural items. For example, at least one (of) a, b, or c may be expressed as: a, b, c, a-b, a-c, b-c, or a-b-c, wherein a, b, and c may be single or multiple.

The present application provides an intermittent hypoxic-hyperoxic training device for both dynamic and static use, a training method, and a terminal. The training device has multiple training modes. The training device includes: a gas generating device, a control device, an interactive display device, a heart rate wearing device, and a blood oxygen saturation detection device. The control device preliminarily sets an oxygen content parameter of a mixed gas upon receiving a corresponding training instruction sent by the interactive display device in response to the training mode selected by the user instruction; a heart rate parameter of a trained object is acquired from the heart rate wearing device, so as to adjust the oxygen content parameter under an exercise state according to the heart rate parameter; and/or a blood oxygen parameter of the trained object is acquired from the blood oxygen saturation detection device, so as to adjust the oxygen content parameter under a non-exercise state according to the blood oxygen parameter. The training device of the present application can be applied to various states of the user, and can flexibly and stably adjust the gas composition and output a stable airflow.

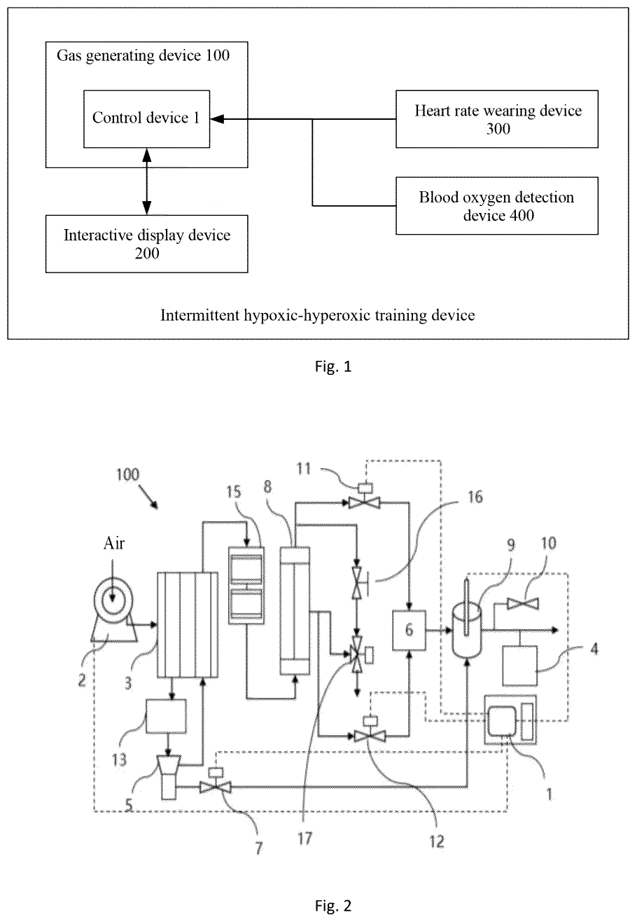

In order to facilitate the understanding of the embodiments of the present application, the embodiments are first described in detail in combination with FIG. 1. FIG. 1 shows a structural schematic diagram of an intermittent hypoxic-hyperoxic training device for both dynamic and static use in an embodiment of the present application. The intermittent hypoxic-hyperoxic training device for both dynamic and static use in the present embodiment is equipped with multiple training modes, and the training device mainly includes a training device body, an interactive display device 200, a heart rate wearing device 300, and a blood oxygen saturation detection device 400.

A gas generating device 100 and a control device 1 are provided inside the training device body.

The interactive display device 200 is arranged on a surface of the training device body to send a corresponding training instruction to the control device 1 in response to a training mode selected by a user instruction to preliminarily set an oxygen content parameter of the mixed gas.

The heart rate wearing device 300 is electrically connected to the control device 1. The heart rate wearing device 300 is worn on a trained object for collecting a heart rate parameter of the trained object.

The blood oxygen saturation detection device 400 is electrically connected to the control device 1. The blood oxygen saturation detection device 400 is worn on the trained object for collecting a blood oxygen parameter of the trained object.

The control device 1 acquires the heart rate parameter of the trained object from the heart rate wearing device 300 for adjusting the oxygen content parameter under an exercise state according to the heart rate parameter; and/or, the control device 1 acquires the blood oxygen parameter of the trained object from the blood oxygen saturation detection device 400 for adjusting the oxygen content parameter under a non-exercise state according to the blood oxygen parameter.

It should be noted that those skilled in the art may select models of the heart rate wearing device 300 and the blood oxygen saturation detection device 400 according to actual needs, which is not limited in the present invention.

In an embodiment, the types of training modes include: a hyperoxic mode, a hypoxic mode, and an artificial mode. Each of the training modes, except for the artificial mode, is preset with a corresponding oxygen content parameter; in the artificial mode, the oxygen content parameter of the mixed gas can be set artificially by the interactive display device 200.

In an embodiment, the training mode of the training device is selected by means of interaction with the interactive display device 200.

Specifically, if a training mode is selected on the interactive display device 200, the interactive display device 200 sends a training instruction corresponding to the training mode to the control device 1, and the control device 1 controls the gas generating device 100 according to the oxygen content parameter set in the training mode after receiving the training instruction, such that the gas generating device 100 produces a mixed gas that satisfies the oxygen content parameter in the training mode.

In an embodiment, the interactive display device 200 is further configured to display parameters related to the training device; the parameters related to the training device include: gas parameters, physiological monitoring parameters, and training device monitoring parameters; and the training device monitoring parameters include: a fluid device temperature monitoring parameter and a pipe pressure monitoring parameter.

In an embodiment, the gas parameters include: an oxygen content parameter, a gas flow parameter, an air pressure parameter, and a humidity parameter.

In an embodiment, the gas parameters may be configured via the interactive display device 200.

In an embodiment, the training device is a training device for both dynamic and static use. The training device may adjust the oxygen content parameter based on the collected physiological parameters of the trained object during training, and this process is completed with the assistance of the heart rate wearing device 300 and the blood oxygen saturation detection device 400. Specifically, since the blood oxygen saturation detection device 400 generally adopts optical components, the measurement of blood oxygen during exercise is inaccurate, so when the trained object is under an exercise state, the heart rate parameter of the trained object is acquired in real time using the heart rate wearing device 300 and is sent to the control device 1, and the control device 1 adjusts the oxygen content parameter according to the heart rate parameter of the trained object, such that the gas generating device 100 produces a mixed gas that is applicable to the current state of the trained object. When the trained object is under a non-exercise state, the blood oxygen parameter of the trained object is collected in real time by the blood oxygen saturation detection device 400 and is sent to the control device 1, and the control device 1 adjusts the oxygen content parameter according to the blood oxygen parameter of the trained object, such that the gas generating device 100 generates a mixed gas applicable to the current state of the trained object.

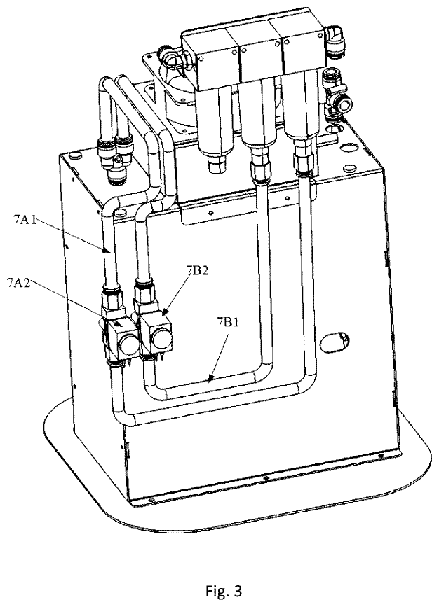

In an embodiment, as shown in FIG. 2, the gas generating device 100 includes: a fluid device 2, a purifying device 15, a gas separating device 8 and a gas mixing device 6 connected in sequence; the fluid device 2 is connected to a gas-liquid separator 5 through a heat exchanger 3, the purifying device 15 is connected to the gas-liquid separator 5 through the heat exchanger 3, and the gas-liquid separator 5 is connected to a drainage structure 7.

In an embodiment, as shown in FIG. 2, the fluid device 2 adopts an air compressor or a blower. Preferably, the fluid device selects an oil-free air compressor or an oil-free blower, and the oil-free air compressor does not use lubricating oil in the process of compressing air, therefore, the compressed air is cleaner, purer and has the advantages of energy saving and environmental protection, and the oil-free blower can also provide oil-free air and is energy-saving.

In an embodiment, as shown in FIG. 2, the fluid device 2 is connected to a condenser 13 via the heat exchanger 3, and the condenser 13 is connected to the gas-liquid separator 5. Specifically, the compressed air output from the fluid device 2 is cooled by the heat exchanger 3, and then is cooled by the condenser 13 to condense the water vapor, and then gas is separated by the gas-liquid separator 5. The cooled dry compressed gas flows from the gas-liquid separator 5 to the heat exchanger 3 and exchanges heat with the hot air in the heat exchanger 3, and the warmed dry compressed air enters the purifying device 15 to remove impurities such as particulate matters.

In an embodiment, the gas-liquid separator 5 is internally provided with a filter element. Preferably, the filter element has a filtration precision of 5 microns.

In an embodiment, the purifying device 15 includes a primary filter element and a secondary filter element. Preferably, the filtration precision of the primary filter element is 0.5 micron and the filtration precision of the secondary filter element is 0.01 micron.

In an embodiment, as shown in FIG. 2, the gas purified by the purifying device 15 flows into the gas separating device 8, the first outlet of the gas separating device 8 outputs nitrogen-rich gas, and the second outlet of the gas separating device 8 outputs oxygen-rich gas. The first outlet is connected to the gas mixing device 6 via a first valve 11, and the second outlet is connected to the gas mixing device 6 via a second valve 12; and the gas mixing device 6 is configured to mix the gas output from the two outlets respectively.

In an embodiment, the gas separating device 8 uses an organic polymer membrane assembly. Preferably, a hollow fiber membrane assembly is used, and the hollow fiber membrane has good durability and can maintain performance in long-term use.

In an embodiment, the gas mixing device 6 may be a section of pipe including a tee joint, or a static mixer may also be arranged in the pipe.

In an embodiment, the first valve 11 is a flow regulating valve. Preferably, an electromagnetic proportional valve is used.

In an embodiment, as shown in FIG. 2, the first outlet is further connected to a third valve 16. The third valve 16 is a pressure regulating valve, preferably a relief valve. When the air pressure at the first outlet exceeds a preset value, the third valve 16 opens and discharges gas, and the second outlet is connected to the atmosphere via a fourth valve 17. The fourth valve 17 is an electromagnetic valve, preferably a two-position three-way valve.

In an embodiment, the gas mixing device 6 is further connected to an oxygen sensor for measuring in real time the oxygen content of the mixed gas and sending the oxygen content to the control device 1.

In an embodiment, as shown in FIG. 3, the drainage structure 7 includes: a first drainage pipe 7A1 and a second drainage pipe 7B1; a first drainage valve 7A2 is arranged on the first drainage pipe 7A1, and a second drainage valve 7B2 is arranged on the second drainage pipe 7B1.

It should be noted that the drainage structure 7 can improve drainage efficiency and also facilitate maintenance, since one drainage pipe can be closed without affecting the use of the other drainage pipe.

In an embodiment, the control device 1 may be integrated in the gas generating device 100 as shown in FIG. 2. The control device 1 may also be independent of the gas generating device, which is not limited in the present invention.

In an embodiment, as shown in FIG. 2 and FIG. 3, the control device 1 is communicatively connected to the first valve 11, the second valve 12, the third valve 16, the fourth valve 17, the first drainage valve 7A2, and the second drainage valve 7B2, respectively; the control device 1 adjusts gas parameters of the mixed gas produced by the gas generating device 100 by controlling the first valve 11, the second valve 16, the first drainage valve 7A2, and the second drainage valve 7B2, respectively.

Specifically, the oxygen content, the gas flow, and air pressure of the mixed gas can be adjusted by controlling the first valve 11 and the second valve 12, and the humidity of the mixed gas can be adjusted by controlling the first drainage valve and the second drainage valve.

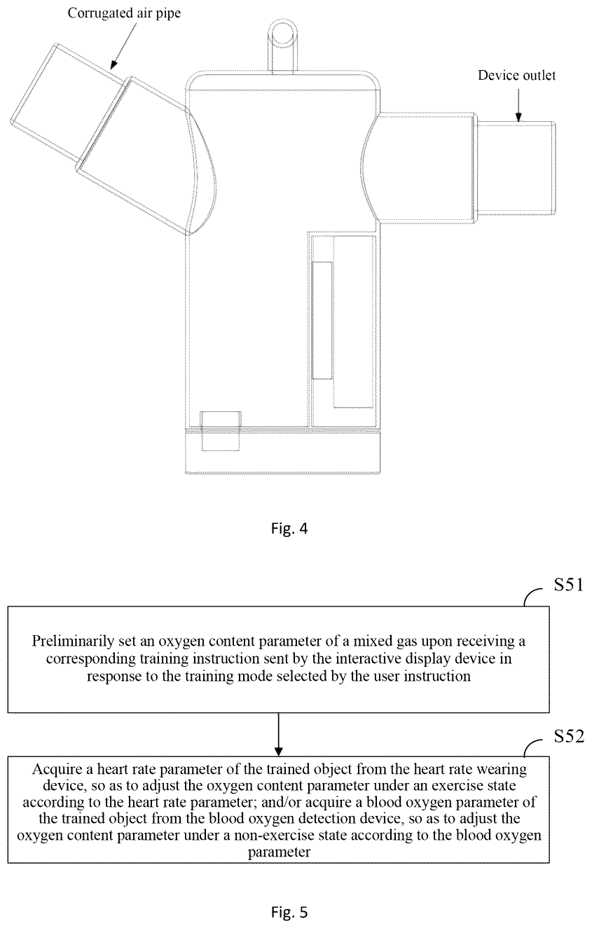

In an embodiment, the training device is further equipped with an external atomizing device 9 as shown in FIG. 4; the external atomizing device 9 is connected to the first drainage pipe, the second drainage pipe, and the gas mixing device 6 of the drainage structure 7 as shown in FIG. 2, respectively, for adjusting the humidity of the mixed gas produced by the gas mixing device 6; the external atomizing device 9 can use the water produced by the gas-liquid separator 5, or can use the liquid stored in the atomizer; and the liquid stored in the atomizer can include: cordyceps, saline, and aroma.

In an embodiment, the external atomizing device is connected to the atmosphere via a fifth valve 10. The fifth valve 10 may be an electromagnetic valve.

In an embodiment, as shown in FIG. 2 and FIG. 4, the outlet of the external atomizing device 9 is also connected to a gas bag 4, and the gas bag 4 can accommodate a certain capacity of oxygen-containing gas, such that when the trained object wears a respiratory mask to use and inhale gas, the gas in the gas bag 4 plays a buffering role. The capacity of the gas bag 4 may range from 2 to 5 L. Since in the present invention, oxygen-containing gas can be continuously output for personal use, no large-capacity buffer gas tank is required.

In an embodiment, the control device 1 is deployed with a well-trained gas parameter optimization model; and the gas parameter optimization model optimizes use parameters for the trained object based on characteristics and preferences of the trained object.

In an embodiment, the training process of the gas parameter optimization model includes:

First, the gas parameter optimization model may acquire basic data, exercise habit data, physiological index data, environmental parameter data and historical use parameter data of the trained object, and form a data set of the trained object after pre-processing all the acquired data.

The gas parameter optimization model may acquire the data by using sensors. The basic data of the trained object may include, but are not limited to, the age, gender, weight, height, health condition, and other data of the trained object; the exercise habit data may include, but are not limited to, the exercise frequency, exercise type, exercise intensity, duration, and other data of the trained object involved in the process of using the intermittent hypoxic-hyperoxic training device in the past; the physiological index data include but are not limited to the heart rate, blood pressure, oxygen saturation, respiratory rate and other data of the trained object; the environmental parameter data include but are not limited to altitude, oxygen concentration, temperature, humidity and the like during training; and the historical use parameter data refer to the parameters of the intermittent hypoxic-hyperoxic training device set by the trained object during previous training.

Pre-processing methods may include, but are not limited to: missing value processing, abnormal value detection, data normalization, data cleaning, etc. The missing value processing refers to deleting records containing missing values and filling in the missing values using predictive values such as mean or median. The abnormal value detection refers to identifying abnormal values using box plots, standard deviation, or quantile-based methods, and dealing with abnormal values by means of deletion, replacement, or transformation. The data normalization refers to the scaling of data to between 0 and 1. The data cleaning means removing duplicate records and correcting errors and inconsistent data.

Second, the gas parameter optimization model divide the data set of the user into a training set, a validation set, and a test set according to a preset ratio, select a machine learning model and an initializing model parameter, input the training set into the machine learning model for training, calculate a prediction by forward propagation, update the model parameters by back propagation, use the validation set to adjust parameters of a model, and evaluate a generalization ability of the model using the test set.

The parameters of the model are adjusted using the validation set, that is, hyper-parameters of the model are adjusted, such as learning rate and regularization strength, and different model structures are selected. The validation set helps to evaluate the performance of the model on unseen data, thereby avoiding overfitting and helping to select the best model configuration. On the other hand, the test set is configured to finally assess the generalization ability of the model, once the model has been trained on the test set and parameters have been adjusted on the validation set, the test set is configured to assess the final performance of the model.

It should be understood that the forward propagation process means that the model receives the training set and generates predicted outputs through a series of calculations (e.g., linear combinations, activation functions, etc.), wherein the data are transmitted layer by layer to an output layer from an input layer. The difference between the predicted outputs of the model and a true label is calculated using a loss function (e.g., mean square error, cross entropy, etc.). The back propagation process means that the gradient of each parameter is calculated using the gradient information of the loss function through a back propagation algorithm, and this process starts from the output layer and transmits to the input layer in reverse, aiming at recruiting partial derivatives of the loss function with respect to each parameter. Subsequently, parameters of a model are updated according to calculated gradients using an optimization algorithm (e.g., gradient descent, Adam, etc.), wherein the magnitude of update is determined by the learning rate, and the learning rate is a hyper-parameter. The above process is repeated for iterating the training, until the performance of the model on the training set reaches a satisfactory level, or a preset number of iterations are reached.

In an embodiment, the gas parameter optimization model optimizes the use parameters for the trained object based on the characteristics and preferences of the trained object.

Specifically, the gas parameter optimization model may identify identity information of the current trained object to acquire characteristics and preferences of the current trained object, input the acquired characteristics and preferences of the current trained object into the well-trained gas parameter optimization model for calculation, and output use parameters applicable to the current trained object; the use parameters include: an oxygen content parameter, training periods, total training time, number of periods, rest time, training intensity, training mode, and user interface parameters.

It should be understood that the type of identity information of the trained object includes, but is not limited to, ID information and the like. The control device is internally stored with characteristics and preferences of multiple trained objects, and matches the characteristics and preferences of the current trained object among the characteristics and preferences of the multiple trained objects via the identity information of the current trained object. The characteristics and preferences of the current trained object include: basic data of the current trained object, exercise habit data, physiological index data, environmental parameter data, and historical use parameter data. Oxygen concentration includes the percentage of oxygen in a hypoxic phase (e.g., 12%-15% of oxygen concentration to simulate a high-altitude environment), and the oxygen concentration in a normoxic phase (usually 21%, that is, the oxygen concentration at sea level). The training period refers to the duration of each hypoxic phase or normoxic phase. The total training time refers to the duration of the entire training talkback. The number of periods refers to the number of alternations between the hypoxic phase and the normoxic phase during a training session. The rest time refers to the rest time between the hypoxic phase and the normoxic phase. The training intensity refers to the intensity of exercise during training. The training mode refers to a preset training mode or a customized mode, and the preset training mode or the customized mode is adjusted according to different training targets and user preferences. The user interface parameters refer to parameters related to the user interface such as display settings, sound cues and visual feedback.

Similar to the above embodiment, as shown in FIG. 5, the present invention further provides an intermittent hypoxic-hyperoxic training method. The intermittent hypoxic-hyperoxic training method is applied to an intermittent hypoxic-hyperoxic training device for both dynamic and static use. The training device has multiple training modes. The training device includes an interactive display device, a gas generating device, a heart rate wearing device, and a blood oxygen saturation detection device. The method includes:
step 51: preliminarily setting an oxygen content parameter of a mixed gas upon receiving a corresponding training instruction sent by the interactive display device in response to the training mode selected by the user instruction; and
step 52: acquiring a heart rate parameter of the trained object from the heart rate wearing device, so as to adjust the oxygen content parameter under an exercise state according to the heart rate parameter; and/or acquiring a blood oxygen parameter of the trained object from the blood oxygen saturation detection device, so as to adjust the oxygen content parameter under a non-exercise state according to the blood oxygen parameter.

It should be understood that the specific processes of the steps in the present embodiment have been described in detail in the above embodiment of the training device and will not be repeated redundantly herein for the sake of brevity.

In an embodiment, the gas generating device includes: a fluid device, a purifying device, a gas separating device and a gas mixing device connected in sequence. The fluid device is connected to a gas-liquid separator through a heat exchanger, the purifying device is connected to the gas-liquid separator through the heat exchanger, the gas-liquid separator is connected to a drainage structure; the gas separating device is provided with a first outlet and a second outlet; the first outlet is connected to the gas mixing device via a first valve, and the second outlet is connected to the gas mixing device via a second valve.

In an embodiment, the drainage structure includes: a first drainage pipe and a second drainage pipe; the first drainage pipe is provided with a first drainage valve, and the second drainage pipe is provided with a second drainage valve.

In an embodiment, the training device is further equipped with an external atomizing device; the external atomizing device is connected to the first drainage pipe, the second drainage pipe, and the gas mixing device, respectively, to adjust a humidity parameter of the mixed gas; the external atomizer is internally stored with liquid; and the liquid includes: cordyceps, saline, and aroma.

In an embodiment, the control device is communicatively connected to the first valve, the second valve, the first drainage valve, and the second drainage valve, respectively; and the control device adjusts the oxygen content parameter and the humidity parameter by controlling the first valve, the second valve, the first drainage valve, and the second drainage valve.

In an embodiment, the control device is deployed with a well-trained gas parameter optimization model; and the gas parameter optimization model optimizes use parameters for the trained object based on characteristics and preferences of a trained object.

In an embodiment, the training process of the gas parameter optimization model includes: acquiring basic data, exercise habit data, physiological index data, environmental parameter data, and historical use parameter data of the trained object, and forming a data set of the trained object after pre-processing all the acquired data; and dividing the data set of the trained object into a training set, a validation set, and a test set according to a preset ratio; selecting a machine learning model and initializing model parameters; inputting the training set into the machine learning model for training, calculating a prediction by forward propagation, and updating the model parameters by back propagation; using the validation set to adjust parameters of a model, and evaluating a generalization ability of the model using the test set.

In an embodiment, the gas parameter optimization model optimizes the use parameters for the trained object based on the characteristics and preferences of the trained object, and the specific process includes: identifying identity information of the current trained object to acquire characteristics and preferences of the current trained object; inputting the acquired characteristics and preferences of the current trained object into the well-trained gas parameter optimization model for calculation, and outputting use parameters applicable to the current trained object; the use parameters include: an oxygen content parameter, training period, total training time, number of periods, rest time, training intensity, training mode, and user interface parameters.

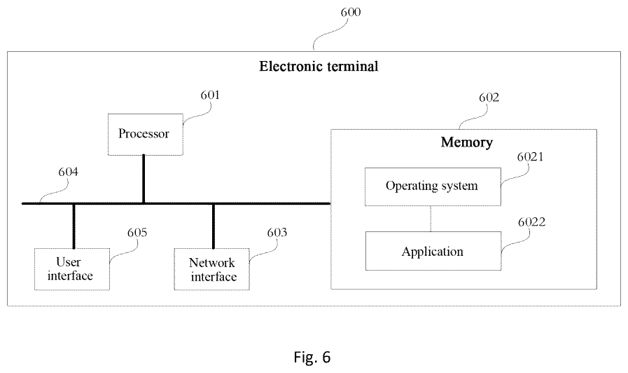

FIG. 6 is a schematic block diagram of an electronic terminal provided by an embodiment of the present application. As shown in FIG. 6, the computer device includes: at least one processor 601, a memory 602, at least one network interface 603, and a user interface 605. Various components in the device are coupled together via a bus system 604. It can be understood that the bus system 604 is configured to enable connected communication between these components. The bus system 604 includes a power bus, a control bus, and a status signal bus in addition to a data bus. However, for clarity of illustration, various buses are labeled as bus systems in FIG. 6.

The user interface 605 may include a display, a keyboard, a mouse, a trackball, a click gun, keys, buttons, a touch pad, or a touch screen.

It can be understood that the memory 602 may be a volatile memory or a non-volatile memory, or may include both a volatile memory and a non-volatile memory. The non-volatile memory may be a read-only memory (ROM), a programmable read-only memory (PROM), which is used as an external cache. Through exemplary but not limiting description, many forms of RAMs are available, such as static random access memory (SRAM) and synchronous static random access memory (SSRAM). The memories described in the embodiment of the present invention are intended to include, but are not limited to, these and any other suitable types of memories.

The memory 602 in the embodiment of the present invention can store various types of data to support operation of an electronic terminal 600. Examples of such data include: any executable program for operation on the electronic terminal 600, such as an operating system 6021 and an application 6022; the operating system 6021 includes various system programs, such as a framework layer, a core library layer, a driver layer, and the like, for implementing various basic services and for handling hardware-based tasks. The application 6022 may contain various applications, such as a media player and a browser and the like, for implementing various application businesses. Implementing the intermittent hypoxic-hyperoxic training method provided by the embodiment of the present invention may be included in the application 6022.

The method disclosed in the above embodiments of the present invention may be applied to the processor 601, or may be implemented by the processor 601. The processor 601 may be an integrated circuit chip with a signal processing capability. During implementation, the steps of the above method may be accomplished by integrated logic circuits of hardware in the processor 601 or by instructions in the form of software. The above processor 601 may be a general-purpose processor, a digital signal processor (DSP), or other programmable logic devices, a discrete gate or a transistor logic device, a discrete hardware component, and the like. The processor 601 may implement or perform various methods, steps, and logic block diagrams disclosed in the embodiments of the present invention. The general-purpose processor 601 may be a microprocessor or any conventional processor. The steps of the accessory optimization method provided in combination with embodiments of the present invention may be directly embodied as being performed by a hardware decoding processor, or performed with a combination of hardware and software modules in a decoding processor. The software module may be located in a storage medium, the storage medium is located in a memory, and the processor reads information in the memory and completes the steps of the above method in combination with its hardware.

In exemplary embodiments, the electronic terminal 600 may be used by one or more application specific integrated circuits (ASICs), DSPs, programmable logic devices (PLDs), complex programmable logic devices (CPLDs) to perform the above method.

The terms “component”, “module”, “system” and the like used in the present specification are used for denoting a computer-related entity, hardware, firmware, a combination of hardware and software, software, or software in execution. For example, components may be, but are not limited to, processes, processors, objects, executables, threads of execution, programs, and/or computers running on a processor. By way of illustration, applications running on a computing device and a computing device may be components. One or more components may reside in processes and/or threads of execution, and the components may be located on a single computer and/or distributed between two or more computers. In addition, these components may be executed from various computer-readable media having various data structures stored thereon. The components may communicate, for example, through local and/or remote processes according to signals having one or more data packets (e.g., data from two components interacting with another component in a local system, distributed system, and/or inter-network, e.g., the internet interacting with other systems via signals).

Those skilled in the art may realize that various illustrative logical blocks and steps described in combination with the embodiments disclosed herein are capable of being implemented in electronic hardware, or a combination of computer software and electronic hardware. Whether these functions are performed in hardware or software depends on the particular application and design constraints of the technical solution. Those skilled in the art may use different methods to implement the described functions for each particular application, but such implementations should not be considered to go beyond the scope of the present application.

Those skilled in the art can clearly understand that, for the convenience and brevity of description, the specific working processes of the systems, devices and units described above can be referred to the corresponding processes in the above method embodiments, and will not be repeated redundantly herein.

In the several embodiments provided in the present application, it should be understood that the systems, devices and methods disclosed may be implemented in other ways. For example, the device embodiments described above are merely exemplary, e.g., the division of the units, which is merely a logical functional division, may be divided in other ways during actual implementation, e.g., multiple units or components may be combined or may be integrated into another system, or some features may be omitted, or may not be implemented. In addition, mutual coupling or direct coupling or communication connection shown or discussed may be an indirect coupling or communication connection through some interfaces, devices or units, and the coupling or connection may be electrical, mechanical or in other forms.

The units illustrated as separated components may or may not be physically separated, and the components displayed as units may or may not be physical units, i.e., they may be located in one place or may also be distributed to multiple network units. Some or all of these units may be selected according to practical needs to fulfill the object of the solution of the present embodiment.

In addition, the functional units in various embodiments of the present application may be integrated in a single processing unit, or each unit may be physically present separately, or two or more units may be integrated in a single unit.

In the above embodiments, the functions of each functional unit may be implemented in whole or in part by software, hardware, firmware, or any combination thereof. When implemented using software, the functions may be implemented in whole or in part in the form of a computer program product. A computer program product consists of one or more computer instructions (programs). When the computer program instructions (programs) are loaded and executed on a computer, the computer program instructions produce, in whole or in part, a process or function according to embodiments of the present application. The computer may be a general-purpose computer, a special-purpose computer, a computer network, or other programmable devices. The computer instructions may be stored in a computer-readable storage medium or transmitted from one computer-readable storage medium to another computer-readable storage medium, e.g., the computer instructions may be transmitted from a web site, a computer, a server, or a data centre via wired (e.g., coaxial cable, optical fiber, digital subscriber line (DSL)) or wireless (e.g., infrared, wireless, microwave, etc.) manner to another website site, computer, server, or data centre. A computer-readable storage medium may be any usable medium to which a computer has access or a data storage device such as a server, a data centre and the like that is integrated by one or more usable media. The usable medium may be a magnetic medium, (e.g., a floppy disk, a hard disk, a tape), an optical medium (e.g., a high-density digital video disc (DVD), or a semiconductor medium (e.g., a solid state disk (SSD)).

Functions may be stored in a computer-readable storage medium if functions are implemented in the form of a software functional unit and sold or used as a separate product. Based on this understanding, the technical solution of the present application essentially or the part contributing to the prior art or the part of the technical solution may be embodied in the form of a software product. The computer software product is stored in a storage medium and includes a number of instructions to cause a computer device (which may be a personal computer, a server, or a network device, etc.) to carry out all or part of the steps of the methods of various embodiments of the present application. The above storage medium includes a USB flash drive, a removable hard disk, a read-only memory (ROM), a random access memory (RAM), a magnetic disc or a compact disc, and other media that can store program codes.

The above is merely a specific embodiment of the present application, but the protection scope of the present application is not limited thereto, and those skilled in the art can easily think of changes or substitutions within the scope of the technology disclosed in the present application, and such changes or substitutions shall fall within the protection scope of the present application.

In summary, the present application provides an intermittent hypoxic-hyperoxic training device for both dynamic and static use, a training method, and a terminal. The training device is equipped with multiple training modes, and the training device includes: a gas generating device, a control device, an interactive display device, a heart rate wearing device, and a blood oxygen saturation detection device. The control device preliminarily sets an oxygen content parameter of a mixed gas upon receiving a corresponding training instruction sent by the interactive display device in response to the training mode selected by the user instruction; a heart rate parameter of a trained object is acquired from the heart rate wearing device, so as to adjust the oxygen content parameter under an exercise state according to the heart rate parameter; and/or a blood oxygen parameter of the trained object is acquired from the blood oxygen saturation detection device, so as to adjust the oxygen content parameter under a non-exercise state according to the blood oxygen parameter. The training device of the present application can be applied to various states of the user, and can flexibly and stably adjust the gas composition and output a stable airflow. Therefore, the present application effectively overcomes various drawbacks in the prior art and has high industrial utilization value.

The above embodiments are merely illustrative of the principles and effects of the present application, and are not intended to limit the present application. Those skilled in the art may modify or change the above embodiments without departing from the spirit and scope of the present application. Therefore, all the equivalent modifications or changes made by those skilled in the art without departing from the spirit and technical ideas disclosed in the present application shall all fall within the claims of the present application.

1. An intermittent hypoxic-hyperoxic training device for both dynamic and static use, wherein the training device is equipped with a plurality of training modes, wherein the training device comprises:
a training device body, wherein a gas generating device for generating a mixed gas and a control device are provided inside the training device;
an interactive display device arranged on a surface of the training device body to send a corresponding training instruction to the control device in response to a training mode selected by a user instruction, so as to preliminarily set an oxygen content parameter of the mixed gas;
a heart rate wearing device electrically connected to the control device, wherein the heart rate wearing device collects a heart rate parameter; and
a blood oxygen saturation detection device electrically connected to the control device, wherein the blood oxygen saturation detection device collects a blood oxygen parameter;
wherein the control device acquires the heart rate parameter from the heart rate wearing device for adjusting the oxygen content parameter under an exercise state according to the heart rate parameter; and/or
wherein the control device acquires the blood oxygen parameter from the blood oxygen saturation detection device for adjusting the oxygen content parameter under a non-exercise state according to the blood oxygen parameter;
wherein the gas generating device comprises: a fluid device, a filter, an organic polymer membrane assembly, and a section of pipe comprising a tee joint connected in sequence;
wherein the fluid device is connected to a gas-liquid separator through a heat exchanger, and the filter is connected to the gas-liquid separator through the heat exchanger, such that the gas-liquid separator is connected between the fluid device and the filter, and the heat exchanger is arranged in both upstream and downstream flow paths of the gas-liquid separator.

2. The intermittent hypoxic-hyperoxic training device for both dynamic and static use according to claim 1, wherein the gas-liquid separator is connected to a drainage structure;
wherein the organic polymer membrane assembly is provided with a first outlet and a second outlet; and
wherein the first outlet is connected to the section of pipe comprising the tee joint via a first valve, and the second outlet is connected to section of pipe comprising the tee joint via a second valve.

3. The intermittent hypoxic-hyperoxic training device for both dynamic and static use according to claim 2, wherein the drainage structure comprises:
a first drainage pipe and a second drainage pipe;
wherein the first drainage pipe is provided with a first drainage valve, and the second drainage pipe is provided with a second drainage valve.

4. The intermittent hypoxic-hyperoxic training device for both dynamic and static use according to claim 3, wherein the training device is further equipped with an external atomizing device;
wherein the external atomizing device is connected to the first drainage pipe, the second drainage pipe, and the section of pipe comprising the tee joint, respectively, to adjust a humidity parameter of the mixed gas;
wherein the external atomizer is internally stored with liquid; and wherein the liquid comprises: cordyceps, saline, and aroma.

5. The intermittent hypoxic-hyperoxic training device for both dynamic and static use according to claim 3, wherein the control device is communicatively connected to the first valve, the second valve, the first drainage valve, and the second drainage valve, respectively; and
wherein the control device adjusts the oxygen content parameter and the humidity parameter by controlling the first valve, the second valve, the first drainage valve, and the second drainage valve.

6. The intermittent hypoxic-hyperoxic training device for both dynamic and static use according to claim 1, wherein the control device is deployed with a trained gas parameter optimization model;
wherein the gas parameter optimization model optimizes use parameters based on user characteristics and preferences of the trained object.

7. The intermittent hypoxic-hyperoxic training device for both dynamic and static use according to claim 6, wherein during a training process, the gas parameter optimization model is configured to:
acquire basic data, exercise habit data, physiological index data, environmental parameter data and historical use parameter data, and form a data set after pre-processing all the acquired data; and
divide the data set into a training set, a validation set, and a test set according to a preset ratio; select a machine learning model and initialize model parameters; input the training set into the machine learning model for training, calculate a prediction by forward propagation, and update the model parameters by back propagation; use the validation set to adjust parameters of a model, and evaluate a generalization ability of the model using the test set.

8. The intermittent hypoxic-hyperoxic training device for both dynamic and static use according to claim 6, wherein the gas parameter optimization model optimizes the use parameters based on the user characteristics and preferences, wherein the gas parameter optimization model is configured to:
identify identity information to acquire user characteristics and preferences, input the acquired user characteristics and preferences into the trained gas parameter optimization model for calculation, and output use parameters;
wherein the use parameters comprise: an oxygen content parameter, training periods, total training time, number of periods, rest time, training intensity, training mode, and user interface parameters.

9. An intermittent hypoxic-hyperoxic training method applied to the intermittent hypoxic-hyperoxic training device for both dynamic and static use according to claim 1, wherein the training device is equipped with the plurality of training modes, wherein the training device comprises the interactive display device, the gas generating device, the heart rate wearing device, and the blood oxygen saturation detection device, wherein the method comprises:
preliminarily setting an oxygen content parameter of the mixed gas upon receiving the corresponding training instruction sent by the interactive display device in response to the training mode selected by the user instruction; and
acquiring the heart rate parameter from the heart rate wearing device, so as to adjust the oxygen content parameter under the exercise state according to the heart rate parameter; and/or acquiring the blood oxygen parameter from the blood oxygen saturation detection device, so as to adjust the oxygen content parameter under the non-exercise state according to the blood oxygen parameter.

10. An electronic terminal, comprising a memory, a processor, and a computer program stored on the memory, wherein the processor executes the computer program to implement the method of claim 9.
